(19) United States
(12) Patent Application Publication
(10) Pub. No.: US 2019/0309015 A1
(43) Pub. Date: Oct. 10, 2019

(54) LIPOSOMAL METHODS FOR PURIFICATION OF PROTEINS

(71) Applicant: SynLife, Inc., Cambridge, MA (US)

(72) Inventor: Felix MOSER, Cambridge, MA (US)

(73) Assignee: SynLife, Inc., Cambridge, MA (US)

(21) Appl. No.: 16/377,989

(22) Filed: Apr. 8, 2019

Related U.S. Application Data
(60) Provisional application No. 62/653,742, filed on Apr. 6, 2018.

(51) Int. Cl. C07K 1/22; G01N 33/543; A61K 39/002; C07K 1/04

(52) U.S. Cl. CPC C07K 1/22; C07K 1/04; A61K 39/002; G01N 33/5432

(57) ABSTRACT

A general method is described for purifying any protein from a complex mixture by anchoring the protein of interest to liposomes that can be easily isolated from the complex mixture, then separated from the liposome and purified if desired.

### CROSS-REFERENCE TO PRIOR APPLICATION

This application claims priority under 35 U.S.C. 119(e) to U.S. provisional patent application Ser. No. 62/653,742, filed Apr. 6, 2018, and is incorporated herein by reference in its entirety.

### BACKGROUND OF THE INVENTION

Cell-free extract is increasingly used to drive transcription/translation (Tx/TL) reactions in vitro for protein production and rapid assays (for example, Carlson et al., Cell-free protein synthesis: applications come of age, Biotechnology Advances 2012 September-October; 30(5):1185-1194). Tx/TL reactions have also shown to be active following freeze-drying and rehydration, enabling on-site production of vaccine antigen or other proteins without need for a cold chain (for example, Pardee et al., Paper-based synthetic gene networks, Cell 2014 Nov. 6; 159(4):940-954; Pardee et al., Portable, on-demand biomolecular manufacturing, Cell 2016 Sep. 22; 167(1):248-259.e12). However, this complex mixture is unsuitable for clinical use and the vaccine or protein of interest must first be purified. In addition to cell-free extract-based protein production, many other protein production processes require purification of the protein before it can be used medically, industrially, for research purposes, among many other uses.

It is towards the facile purification of proteins from Tx/TL or any other protein production systems that the present invention is directed.

### SUMMARY OF THE INVENTION

The invention is generally directed to methods for facilely purifying proteins for therapeutic and other uses. In one embodiment, a method is provided for the purification of a protein comprising the steps of (1) providing the protein in the presence of liposomes, wherein the protein comprises means for binding to the liposomes and the liposomes comprise means for binding to the protein, and wherein the protein binds to the liposomes forming protein-decorated liposomes, and wherein the binding of the protein to the liposomes enables the separability of the protein-decorated liposomes; (2) isolating the protein-decorated liposomes; (3) separating the protein from the protein-decorated liposomes; and (4) purifying the protein from the protein-decorated liposomes.

In one embodiment, the means for binding of the liposomes to the protein comprises a surface moiety on the liposomes that binds to the protein. In one embodiment, the means for protein binding to the liposomes comprises a moiety on the protein that binds to the liposomes. In one embodiment, the moiety is added to the protein or the moiety is expressed with the protein.

In one embodiment, the binding is reversible, irreversible, non-covalent or covalent. In one embodiment, the non-covalent means is selected from nickel-NTA-6×His tag binding, streptavidin/biotin binding, conjugate antibody binding, binding between complementary protein fragments, hybridization of complementary DNAs or RNA and binding to DNA or RNA aptamers. In one embodiment, the protein comprises a His tag and the liposomes comprise a nickel-NTA moiety. In one embodiment, the covalent means is selected from click chemistry and spytag/spycatcher chemistry. In one embodiment,

In one embodiment, the separability is selected from a change in density of the liposomes associated with protein binding, a change in the surface charge of the liposomes, a change in the stiffness or shape of the liposomes, and a change in the fluorescence, luminescence, light absorbance or light emission of the liposomes.

In one embodiment, the liposomes are freeze-dried liposomes and rehydrated before use. In one embodiment, the liposomes comprise phospholipids, cholesterol, conjugated phospholipids, small molecule adjuvants such as QS21 and saponins, cationic lipids internal or external to the liposome, and any combination thereof.

In one embodiment, the isolating is achieved by gravity centrifugation, density centrifugation, extraction, countercurrent distribution, dialysis, chromatography, precipitation, washing or any combination thereof.

In one embodiment, the protein is separated from the protein-decorated liposomes by proteinase cleavage, competitive binding, or a change in pH, ion content or strength, temperature or light exposure.

In one embodiment, the separated protein is purified from the liposomes by gel permeation chromatography, centrifugation, dialysis, filtration, precipitation, chromatography, or any combination thereof.

In one embodiment, the protein is a vaccine antigen, an enzyme, an antibody chain, a signal protein such as insulin and human growth hormone, a viral coat protein or a structural protein such as collagen.

In one embodiment, a protein made by the afore described methods is provided

In one embodiment, a method for the preparation of protein-decorated liposomes comprising the steps of: (1) providing the protein in the presence of liposomes, wherein the protein comprises means for binding to the liposomes and the liposomes comprise means for binding to the protein, and wherein the protein binds to the liposomes forming protein-decorated liposomes, and wherein the binding of the protein to the liposomes enables the separability of the protein-decorated liposomes; and (2) isolating the protein-decorated liposomes.

In one embodiment, a protein-decorated liposome made by aforementioned method is provided. In one embodiment, the protein is a vaccine antigen.

In other embodiments, the invention is directed to a protein-decorated liposome made by the aforementioned methods, and the various embodiments thereof. In one embodiment, the protein-decorated liposomes are therapeutically useful. In one embodiment, the therapeutically useful protein-decorated liposomes comprise a liposome-adjuvanted protein vaccine, a monoclonal antibody, a signaling protein, a viral capsid, a receptor protein or a structural protein.

In one embodiment, the invention is directed to a protein-decorated liposome made by the method described herein. In one embodiment, the invention is directed to a protein made by the methods described herein.

According to some embodiments of the invention, the expression system comprises live cells selected form the group consisting of prokaryotic cells, eukaryotic cells, bacterial cells, fungi cells, yeast cells, algae cells, plant cells, parasite cells, insect cells, animal cells, ovarian cells, fish cells, bird cells and mammalian cells. In some embodiments of the invention, a cell free extract may be prepared or derived from any such cell type.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 depicts one embodiment of the method of the invention;

FIG. 2 shows the selective binding of a target protein to the liposomes of the invention; and

FIG. 3 shows the purification of the target protein after elution from liposomes, and purification from endotoxin.

### DETAILED DESCRIPTION OF THE INVENTION

As used herein the term “about” refers to ±10%.

The terms “comprise”, “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a scaffold” or “at least one scaffold” may include a plurality of scaffolds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term liposomes as embodied herein includes any vesicle or nanoparticle-based systems such as but not limited to particles typically referred to as liposomes, comprising a one or more lipid layers surrounding an aqueous core.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

A general method is described for purifying any protein from a complex mixture by anchoring the protein of interest to liposomes that can be easily separated or isolated from the complex mixture by methods such centrifugal precipitation. In one embodiment, the liposomes have on their surface chemical groups to which the protein of interest can be chemically anchored (decorated) either passively or actively, which enables rapid, robust purification of proteins of interest (e.g. vaccine antigens, enzymes, etc.) from a complex milieu of, for example, active cell-free extract, lysed cells, or other heterogeneous chemical mixtures. This invention enables rapid purification of vaccine formulations such as the RTS,S malaria vaccine, which are formulated with liposome adjuvants and can be improved when antigens are closely associated or bound to liposomal adjuvants (for example, Alving et al., “Liposomal adjuvants for human vaccines”, Expert Opinion on Drug Delivery 2016 Feb. 25; 13(6):807-816; RTS,S Clinical Trials Partnership, “Efficacy and safety of RTS,S/AS01 malaria vaccine with or without a booster dose in infants and children in Africa: final results of a phase 3, individually randomised, controlled trial”, The Lancet 2015 Jul. 4-10; 386(9988):31-45). This method also offers a solution for the need to purify proteins from freeze-dried cell-free reactions. In some embodiments, the vaccine is the protein-decorated liposomes of the invention.

Cell-free extract is increasingly used to drive transcription/translation (Tx/TL) reactions in vitro for protein production and rapid assays (for example, see Carlson et al., “Cell-free protein synthesis: Applications come of age”, Biotechnology 2012 September-October; 30(5):1185-1194; Kuruma et al., The PURE system for the cell-free synthesis of membrane proteins, Nat Protoc 2015 September; 10(9):1328-1344). Tx/TL reactions have also shown to be active following freeze-drying and rehydration, enabling on-site production of vaccine antigen or other proteins without need for a cold chain (for example, see Pardee et al., “Paper-Based Synthetic Gene Networks”, Cell 2014 Nov. 6, 159(4):718-720; Pardee et al., “Portable, On-Demand Biomolecular Manufacturing”, Cell 2016 Sep. 22, 167(1):248-259). However, this complex mixture is unsuitable for clinical use and the vaccine or protein of interest must first be purified from it. In one embodiment of the methods described here, the Tx/TL reaction mixture is added to dehydrated liposomes. The produced protein then binds to the liposomes selectively through either a noncovalent interaction (in one embodiment, 6×His tag) or formation of a covalent bond (in certain embodiments, click chemistry, spycatcher/spytag). In this example, binding of the target protein to the liposomes increases the relative density of the liposomes. This enables the decorated liposomes to be easily pelleted by centrifugation, and subsequent washing removes residual impurities. In the case where the target protein is vaccine antigen, the liposome formulation may then be ready to be injected into the patient. In the case where the target protein needs to be separated from the liposome and further purified, the protein can be removed or separated from the liposome by biochemical means such as but not limited to competitive binding inhibition or protease cleavage. Subsequently, the separated protein can be purified from the liposomes by any of various methods such as gel permeation chromatograph. The aforementioned description is merely one embodiment of the invention, which can be carried out in numerous variations thereof all of which are embraced herein.

As noted above, in one embodiment, this method utilizes the change in density that occurs when liposomes are bound to the protein to facilitate isolation or separation of the protein-decorated liposomes from the rest of the milieu. Without this binding, the lipids are less dense than the surrounding aqueous milieu and will rise to the top of an aqueous solution, making it difficult to remove the mixture. Depending on the target protein being provided and purified by the methods herein, the liposome formulation can be altered to maximize the efficacy of the purification method. In one embodiment the binding of the protein facilitates the density change of the liposomes. In one embodiment the protein of interest in the presence of the reaction mixture facilitates the change in density.

Thus, the invention offers a fast and effective means of purifying proteins to high levels of purity. This is especially useful in one embodiment, for protein production from freeze-dried, cell-free reactions, which can be useful to generate medically-useful protein in remote facilities where maintaining a cold chain is difficult. The procedure also enables rapid production of vaccines composed of liposomes bound to antigen, which may increase the immunogenicity and therefore efficacy of the vaccine. Where the separation of the protein from the liposome is desired, the protein can be separated from the liposome by enzymatic or chemical means designed into the selection of the binding means, then the protein can be isolated or purified from the liposomes by methods well known in the art.

In the following description, each of the steps of the invention and variations thereof are described. This description is not intended to be limiting and changes in the components, sequence of steps, and other variations would be understood to be within the invention.

Proteins of Interest.

As noted above, the utility of the protein of interest is non-limiting, and may be for any medical, diagnostic, industrial, commercial or research purpose. In one embodiment, the protein is a vaccine antigen that can be administered in combination with a liposome to elicit an immune response adjuvanted by the liposome components. In other embodiments, the protein is separated from the liposome, isolated and further purified, for use for its intended purpose.

In one embodiment the protein is a vaccine antigen, an enzyme, an antibody chain, a signal protein, a viral coat protein or a structural protein. In one embodiment, the therapeutically useful protein-decorated liposomes are a liposome-adjuvanted protein vaccine. In one embodiment the protein of interest is a signal protein such as insulin and human growth hormone. In one embodiment the protein of interest is a viral coat protein. In one embodiment the protein of interest is a structural protein such as collagen.

In one embodiment, the method provides the protein of interest by means of expression, which in one embodiment may be a cell-free extract. In one embodiment, the cell-free extract is freeze-dried, such that it can be reconstituted at the time of desired purification of the protein of interest; in one embodiment the liposomes are also freeze dried and reconstituted together with the cell free extract at the time of protein expression and purification, all of which can be accomplished in a “one-pot” system in accordance with the teachings herein. The cell free extract containing the expression system components, nucleic acid encoding the protein of interest (with the necessary liposome-binding means) and the liposomes (with the protein binding means) may be mixed together; once sufficient protein is generated and binds to the liposomes, the protein-decorated liposomes can be pelleted by centrifugation and washed to remove other materials from the reaction matrix. Then the pelleted liposomes with the bound protein can be used for the intended purpose directly, or in some embodiments, the protein my be unbound from the liposome and subsequently isolated or purified therefrom, and the purified protein used for the intended purpose.

Liposomes.

In one embodiment the liposomes are formulated from art-recognized basic components, such as but not limited to the phospholipid POPC and cholesterol. Included in the formulation is the binding moiety for the protein of interest, such as nickel-bound nitrilotriacetic acid or SpyCatcher, as described above. Furthermore, the liposome is formulated to, in one embodiment, change density upon binding to the protein or interest, or in other embodiments, undergo a different physicochemical change that can be exploited to separate protein-bound liposomes from non-bound liposomes and other materials in the matrix. Furthermore, during preparation the liposome may be filled with a material that can facilitate such change in physicochemical property or separation.

“Liposomes” as used here is not limited to the traditional meaning of liposomes but includes any vesicle or nanoparticle-based system that comprises a lipid bilayer phase and an aqueous phase, although even such a description is intended to be non-limiting to the scope and operation of the invention. In other embodiments, liposomes include peptide vesicles, which in one embodiment use amphiphilic peptides instead of phospholipids. Among other embodiments, micelles and ethosomes are embodied herein. As noted elsewhere herein, the separability of the liposome after binding the protein of interest from other components is required, and in one embodiment, a change in density of the liposomes is one change exploited for such separation. However the invention is not limited to any particular change in property or means of separation utilizing that property, nor form of the particle to which the protein of interest is associated, to fall within the intention of the invention.

Preparation of liposomes is well known in the art and liposomes are typically, though not exclusively, composed of phospholipid bilayers. A phospholipid is an amphiphilic molecule typically consisting of a hydrophilic phosphate head group and two hydrophobic lipid chains. Phospholipids compose the majority of cell membranes and self-aggregate into bilayers that make up liposomes. Liposomes can be composed of other amphiphilic molecules. In one embodiment, the density of the liposomes is prepared so that they can be efficiently separated from the reaction solution upon binding to the protein of interest. As will be noted below, the liposomes are, in one embodiment, diluted before separation, and the density of the dilution solution and the density of the liposomes can be prepared to maximally allow separation based on the particular protein of interest, method of providing the protein, the other components of the reaction solution (e. g., the Tx/TL reaction), among other factors. The composition of the liposomes and/or the composition of the diluent will be readily adjusted to exploit the heretofore unrecognized change in liposome density that accompanies decoration by the protein of interest. In other embodiments, such unrecognized changes will be exploited for separation by other methods.

Guidance for the use of cell-free Tx/TL extracts as well as liposomes may also be found in the following: Zemella, A. et al., (2015) ChemBiochem Vol. 16, Issue 17:2420-2431; Forster, A. C. & Church, G. M, (2006) Mol. Syst. Boil 2, 45; Brea, R. J. et al., (2015) Chem. A Eur. J. Vol. 21, Issue 36:12564-12570; Luisi, P. L. et al., (2006) Naturwissenchaften 93, 1-13; Stano, P. & Luisi, P. L. Curr Opin Biotechnol. (2013) 24:633-638; Tan, C. et al. (2013) Nat. Nanotechnol. 8, 602-8; de Souza, T. P. et al. (2012) Orig. Life Evol. Biosph. 42, 421-428; de souza, T. P., et al., (2014) J. Mol. Evol. 79, 179-192; and Caschera, f & Noireauz, V. (2014) Curr. Opin. Chem. Biol. 22, 85-91, each of which is incorporated herein by reference in its entirety. For example, mammalian cell-free TX/TL systems have been developed to synthesize long, complex proteins that require folding chaperones and post-translational modifications (Brödel, A. K. & Kubick, S. (2014) Pharm. Bioprocess. 2, 339-348). In another example, commercially available rabbit reticulocyte systems offer cap-independent translation and contain mammalian folding chaperones. The glycosylation of proteins is possible in this system upon addition of canine pancreatic microsomal membranes. In one embodiment, human HeLa cell extract is also commercially available; it is used to express antibodies, as well as large and complex proteins and viruses [see Machida, K. et al., (2012) Protein Synthesis in vitro: Cell-Free Systems Derived from Human Cells, Cell-Free Protein Synthesis, Prof. Manish Biyani (Ed.), InTech, DOI: 10.5772/48563. Available from: www.intechopen.com/books/cell-free-protein-synthesis/protein-synthesis-in-vitro-cell-free-systems-derived-from-human-cells and Mikami, S. et al., (2008) Protein Expr. Purif. 62, 190-198].

In one embodiment, liposomes can be loaded with small molecules, proteins, DNA, RNA, other amphiphilic lipids, or mixtures thereof. Liposomes loaded with small molecule drugs are common therapeutics known for their advantageous pharmacokinetics. Well-known among these are AmBisome™ (amphotericin B liposomes), the first liposome-based antifungal therapy, and Doxil™ (doxorubicin liposomes), a leukemia drug. Liposomes loaded with cationic lipids and DNA have been used for gene therapy. Recently, Patisiran™, an RNAi liposome formulation, was approved for use as the first liposome-delivered gene therapy. Liposomes can also be loaded with cell-free extract and DNA simultaneously, generating “synthetic minimal cells” that can perform transcription and translation internally to the liposome, such as described in U.S. patent application Ser. No. 15/430,502, published as US-2017-0233748-A1, incorporated herein by reference in its entirety. Any variations on the liposomes are other embodiments of the invention.

Liposome Decoration.

As described above, a method for the purification of a protein of interest is provided comprising the steps of providing the protein in the presence of liposomes, wherein the protein binds to the liposomes forming protein-decorated liposomes, and wherein the binding of the protein to the liposomes enables the separability of the protein-decorated liposomes; and subsequently isolating the protein-decorated liposomes. The invention can be carried out with different variations without deviating from the general principle of the invention. In its general form, the protein that is to be purified comprises means for binding to the liposomes and the liposomes comprise means for binding to the protein. This may be achieved in any number of ways, simply requiring the desired protein when in contact with the liposome to bind to the liposomes such that the protein-decorated liposomes can be isolated from the matrix in which the binding has occurred. This binding may be non-covalent or covalent, and it may or not be reversible. If the protein is desired to be isolated from the liposomes, the linkage should be reversible. Both noncovalent and covalent binding may be reversible.

In one embodiment, the liposomes are prepared such that they include a moiety on the surface that binds to the protein. In some embodiments the moiety will bind to the native protein, that is, without any requirement of also modifying or altering the protein of interest in order that it may bind to the liposomes. In this embodiment, the liposome is modified to contain a moiety that specifically binds to the protein of interest. Non-limiting examples of such moieties on the liposome include a ligand, receptor, metal, substrate, antibody and antibody ligand. In other embodiments, the protein is modified to contain a moiety that can bind the liposome. In some embodiments, the moiety does not interfere with the biological activity or property or properties of the protein. In some embodiments the modification of the protein is cleaved or otherwise removed in a later step. In one embodiment the moiety is a sequence of amino acids fused into the nucleic acid encoding the protein of interest, such that when the protein is expressed the liposome-binding moiety is expressed in the protein. In one embodiment, the sequence of amino acids is a series of six histidines, known in the art as a His tag, His6 tag, or 6×His tag, which sequence binds non-covalently to nickel atom chelated by a nitrilotriacetic acid (NTA) moiety on the liposome. In some embodiments, these amino acids will be fused to the amino or the carboxy terminal end of the protein, with or without an intervening linker sequence. Other modifications of the protein may include antibody-specific peptide tags (e.g. FLAG tag, myc tag, HA tag, Strep-tag, etc.), conjugation to DNA or RNA that can then be hybridized to a complementary group on the liposome, conjugation to an antibody that then binds the moiety on the liposome, conjugation to various chemical groups that can be selectively bound to a moiety on the liposome, conjugation to protein domains that can bind to complementary protein domains, etc.

As noted above, the liposomes comprise a surface moiety that binds to the protein of interest, either in its native form or with an added moiety to facilitate liposome binding. If a 6×His tag is used to bind the protein, a nickel-bound nitrilotriacetic acid moiety will be formulated with the liposome. Other moieties that can non-covalently (or covalently; see below) bind modified proteins include those described above and known in the art, such as those described by Sunasee and Narain, “Covalent and noncovalent bioconjugation strategies,” in Narain, R., ed., “Chemistry of Bioconjugates: Synthesis, Characterization and Biomedical Applications.” 2014; John Wiley & Sons, Inc., incorporated herein by reference in its entirety.

In addition to non-covalent means for binding the protein of interest to liposomes, covalent methods can also be used. These can be reversible or cleavable, or non-cleavable, depending on the desired further processing or use of the protein. Reversible linkage chemistries include, by way of non-limiting examples, click chemistry and spytag/spycatcher chemistry and those described by Sunasee and Narain, 2014, op. cit. Click chemistry is a term that was introduced by K. B. Sharpless in 2001 to describe reactions that are high yielding, wide in scope, create only byproducts that can be removed without chromatography, are stereospecific, simple to perform, and can be conducted in easily removable or benign solvents. Several types of reaction have been identified that fulfill these criteria, thermodynamically-favored reactions that lead specifically to one product, such as nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael Additions, and cycloaddition reactions. Click chemistry can be used to reversibly bind the protein of interest to the liposome, and later cleave the linkage if desired. Another reversible means of conjugating protein to liposome can be achieved using spycatcher/spytag technology, introduced by Mark Howarth. The SpyTag sequence AHIVMVDAYKPTK can be encoded with the protein of interest, and the expressed protein will form an amide bond with SpyCatcher protein, which can be linked to liposomes.

Isolation of the protein of interest from the liposomes is described below.

Properties of Decorated Liposomes.

In order for the purification method of the invention to be operative, the physicochemical properties of the liposome are modified when the protein of interest is bound. In one embodiment, the relative density of the liposome is changed upon binding of protein thereto, enabling separation by means such as pelleting by centrifugation, differential centrifugation, density centrifugation, and any other methods that facilitate separating the liposomes to which protein is bound from undecorated liposomes any other materials in the matrix in which the binding of the protein to the liposomes takes place. As will be noted below, the formulation of the liposome will take into account this required change in density.

Other changes in the physicochemical properties of decorated versus undecorated liposomes can similarly be exploited in the carrying out of the method of the invention. Properties such as charge, solubility, size, shape, stiffness, color (including absorbance or emission at any wavelength), fluorescence and luminescence can be used to separate decorated from undecorated liposomes. In one embodiment, the liposomes in the reaction mixture undergo the physicochemical change by the lipid component, aqueous component, or both binding, absorbing or solubilizing one or more components present in the reaction mixture. In one embodiment the physicochemical property change in the decorated liposome is mediated by factors such as uptake of components from the reaction mixture into the liposome, change in size or number of lamellae of the liposome, components of the reaction mixture integrating into the lipid bilayer of the liposome, by way of non-limiting examples. The invention is not bound by the means by which the physicochemical properties of the liposomes change upon interacting with the protein of interest in the reaction mixture; in one embodiment the liposomes become isolatable from the reaction mixture as a result of protein of interest binding to the liposomes.

Moreover, in one embodiment, a separation or isolation of liposomes from the rest of the components of the reaction mixture is obtained, such that liposomes decorated to various extents with the protein of interest are separated or isolated from non-liposome components. In one embodiment, all liposomes are separated or isolated from non-liposome components in the reaction mixture. In one embodiment, described below, the protein of interest is subsequently separated then purified from the liposomes. In one embodiment, any components in the aqueous phase of the liposome are not separated and isolated from the liposomes.

As noted above the composition of the liposomes requires the presence of binding means for the protein of interest on the surface, and a physicochemical change in property upon binding to the protein of interest. In one embodiment the liposomes are freeze-dried and rehydrated before use.

Separation or Isolation of Decorated Liposomes.

As noted above, once the protein of interest has bound to and decorated the liposomes, the change in physicochemical properties of the liposome facilitates separation or isolation from the other components of the matrix including, in one embodiment, liposomes with none or too few protein molecules bound to undergo the physicochemical change. In one embodiment all liposomes that undergo a physicochemical change after binding to or being in contact with the reaction mixture are separated or isolated. In the embodiment in which a density change in the liposomes occurs on decoration by the protein of interest, various means of separation by density can be performed to isolate the decorated liposomes from other materials. Methods including pelleting by centrifugation or by density centrifugation can be undertaken. In other embodiments, dialysis, filtration, precipitation or chromatography may be used. These are non-limiting examples. Other examples include fluorescence (such as by fluorescence activated cell sorting of FACS), luminescence, or light absorbance or emission of the liposomes, at any wavelength, including but not limited to ultraviolet, visible and infrared wavelengths.

In one embodiment, the reaction mixture of matrix, in which the liposomes bind to protein of interest, is diluted before separation or isolation. Dilution may be done, in one embodiment, with water or buffer. In one embodiment the dilution step is carried using an aqueous solution of a specific density. In another embodiment, the density of the diluent is prepared to maximize the separability of the liposomes from the reaction solution.

Other changes in the liposome physicochemical properties upon decoration by the protein of interest can be used for separation or isolation. Such methods include extraction, countercurrent distribution, chromatography and immunoprecipitation. In any of the methods used to separate or isolate the desired protein-decorated liposomes, washing steps may be used to further eliminate unwanted materials. Any combination of separation or isolation steps, and washing steps, may be performed to purify the protein-decorated liposomes to the desired level; the material can be tested for purity of the protein of interest and/or presence of undesired contaminants, and further separation steps carried out depending on the level of purity needed. For clinical use, reduction in endotoxin levels is desired.

In one embodiment, the method generates protein-decorated liposomes that are useful for various purposes without in one embodiment further purification or in one embodiment separation of the protein of interest from the liposomes. In one embodiment, the protein-decorated liposomes containing an antigen or immunogen are administered to a subject, to elicit an immune response. In some embodiments the liposome acts as an adjuvant to increase the immunogenicity of the protein antigen. In other embodiments the liposome aqueous phase may be loaded with a therapeutically useful component that works in concert with the protein of interest. In other embodiments, the protein bound to liposomes is useful for industrial processes.

Separation of Protein of Interest from Liposomes.

In some embodiments, the protein of interest can be separated from the liposomes. Non-covalent binding of the protein of interest to the liposomes can be reversed by various means known to disrupt non-covalent binding. In the case of the 6×His-tag-nickel association, the competitive inhibitor imidazole can be used to dissociate the binding. In other embodiments, specific agents can be used to dissociate binding, as are taught in the art. In the embodiments where covalent binding is used to achieve the linkage of the protein of interest to the liposome, enzymatic (e.g., proteinase), light-induced cleavage, or chemical means can be used to break the bond. These are merely non-limiting examples and one of skill in the art will readily identify both the means for binding the protein of interest to liposomes, and means for breaking the covalent bonds or non-covalent association in order to separate then isolate the protein of interest from the liposomes. Reference is made to the Sunasee and Narain 2014 publication, op. cit., for a variety of methods for noncovalently and covalently linking then releasing proteins that are applicable to the invention.

Purification of the Protein.

In one embodiment, once the protein is separated from the liposomes, the protein can be isolated or purified away from the liposomes by any of a number of means. Well known methods include gel exclusion chromatography, extraction of liposome components, dialysis, immunoprecipitation, size-exclusion chromatography, affinity chromatography, ion exchange chromatography, salt-induced precipitation or elution, by way of non-limiting examples. Any of the foregoing methods may be adapted to displace the protein from the liposome. In some embodiments this purification may involve or be referred to as elution from the liposome.

In another embodiment, the invention is directed to a protein-decorated liposome that is made by the methods described herein. In one embodiment the protein-decorated liposome further contains components in the aqueous portion, the lipid portion, or both, that are useful for increasing, enhancing, synergizing or otherwise increasing the utility of the protein of interest bound to the liposome. In one embodiment, the liposome contains, in addition to the bound protein of interest, another component that, when the liposome reaches the environment of interest, interacts with the protein of interest to, in one embodiment activate, in one embodiment deactivate, and in one embodiment synergize with the protein of interest. In one embodiment the interaction results in the location of the liposome being detectable. In one embodiment the environment is a cellular, tissue or organ compartment within an animal, such as a mammalian animal, in one embodiment a human. In one embodiment the environment is an ecological niche. In one embodiment the environment is a component within a machine or fluidic machinery. In one embodiment the environment is within a diagnostic device. In one embodiment the diagnostic device is a human diagnostic device.

In one embodiment the invention is directed to a purified protein made by the process described herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

### Purification of Green Fluorescent Protein (GFP) and the RTS,S Malaria Vaccine Antigen

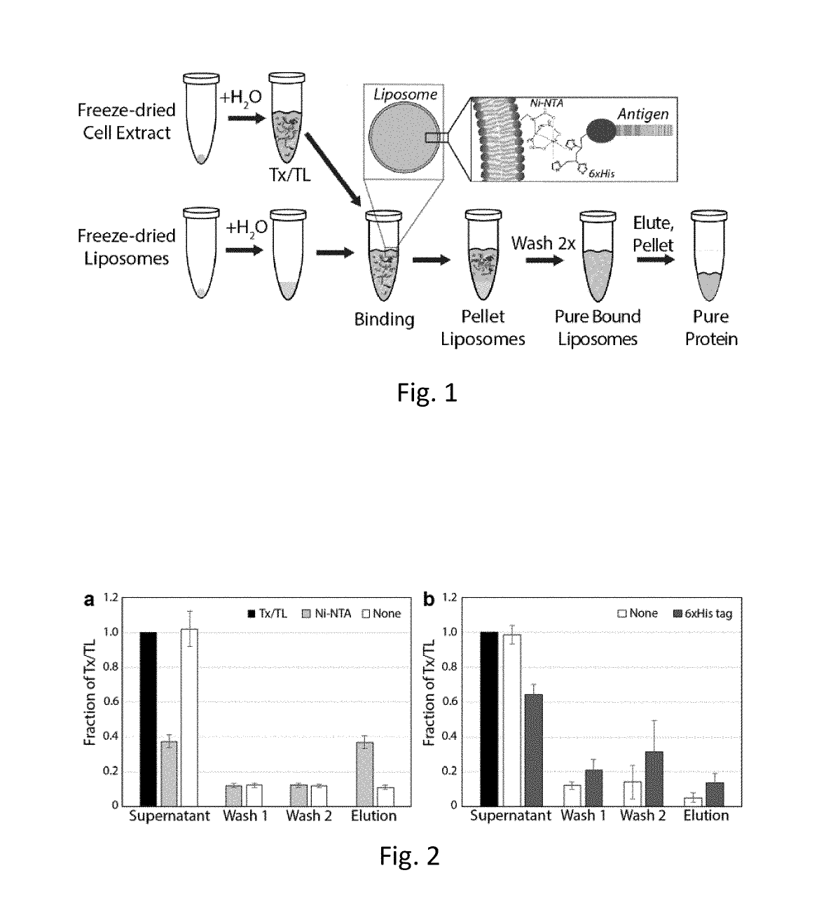

The general method of one embodiment of the invention is shown in FIG. 1, in which liposomes can be used to purify protein from complex samples. In this example, freeze-dried liposomes are used to purify protein produced from a freeze-dried cell extract. Liposomes and extract are combined, enabling the 6×His-tagged product protein to bind the liposomes. The liposomes can then be pelleted and the impurities removed. The final resuspended liposome solution contains liposomes and bound target protein, ready to use as vaccine if the target protein is antigen. If further purification of the target protein is required, the target protein can also be biochemically cleaved from the liposomes and the liposomes removed by pelleting.

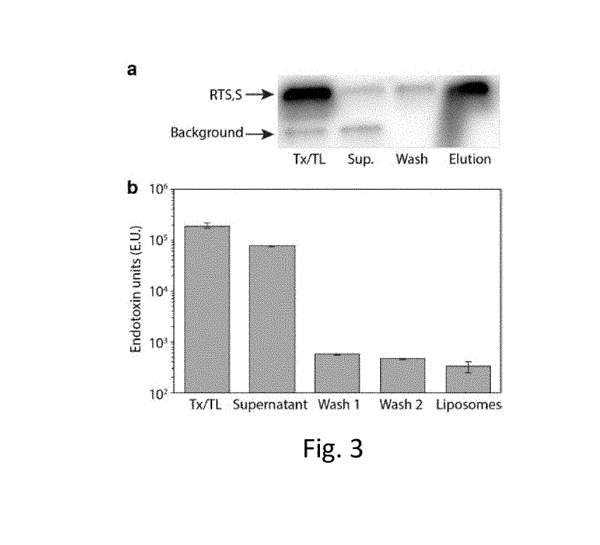

The method was carried out with two exemplary proteins, GFP and the RTS,S malaria vaccine antigen, which were purified from a Tx/TL reaction. To enable purification of GFP and RTS,S from the Tx/TL reaction, these proteins to the 6×Histidine (6×His) peptide tag, which binds strongly to nickel-bound nitrilotriacetic acid (Ni-NTA) groups. Liposomes were prepared composed of the phospholipid POPC, cholesterol, and a phospholipid containing a Ni-NTA head group, and were extruded through a filter to achieve a defined size (1 μm diameter), and freeze-dried. When a crude Tx/TL reaction producing GFP is mixed with these liposomes, the GFP fluorescence in the supernatant of the reaction drops, indicating binding of GFP to the liposomes. This binding was shown to be both 6×His-tag and Ni-NTA dependent (FIG. 2a, b). Western blotting showed that this protocol removed many of the impurities from a Tx/Tl reaction producing RTS,S (FIG. 3a). It also effectively removed endotoxin initially present in Tx/TL reactions, leaving a liposome-protein complex that is suitable for clinical administration (FIG. 3).

FIG. 2 shows the selective purification of GFP was produced in Tx/TL reactions and purified as described. Briefly, 100 μl of the Tx/TL reaction was added to 100 μl of freeze-dried liposomes. Liposomes were then pelleted, washed twice with 1 ml of water, and GFP was eluted with 50 μl of 500 mM imidazole. Samples were taken at each step of the procedure and measured for GFP fluorescence. The measured fluorescence in each sample was divided by the fluorescence of the initial Tx/TL reaction and is plotted as Fraction of Tx/TL. Supernatant is the aqueous fraction isolated immediately after binding to liposomes. (a) Comparison of GFP-6×His binding to liposomes formulated either with (Ni-NTA) or without (none) Ni-NTA phospholipid. (b) Comparison of untagged GFP (none) and 6×-His-tagged GFP (6×His tag) binding to liposomes containing Ni-NTA phospholipid.

In FIG. 3, liposome purification of RTS,S and removal of endotoxin from Tx/TL reaction are shown. In FIG. 3(a), a 6×His-tagged RTS,S antigen was produced in a Tx/TL reaction and purified as described. Samples were taken at each step of the purification and 3 μl of each sample was run on a Western blot with anti-6×His primary antibody. The blot shows the removal of background protein and retention of RTS,S, which can be eluted from the liposomes at high efficiency. In FIG. 3(b), a 6×His-tagged GFP was produced in Tx/TL and bound to Ni-NTA liposomes. The GFP was purified as described in FIG. 2, and samples were taken at each step of the purification. The endotoxin amount in each sample was then measured using an EndoLisa fluorescent assay kit. Endotoxin units (E.U.) were calculated from a standard curve.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for the purification of a protein comprising the steps of:
a) providing the protein in the presence of liposomes, wherein the protein comprises means for binding to the liposomes and the liposomes comprise means for binding to the protein, and wherein the protein binds to the liposomes forming protein-decorated liposomes, and wherein the binding of the protein to the liposomes enables the separability of the protein-decorated liposomes;
b) isolating the protein-decorated liposomes;
c) separating the protein from the protein-decorated liposomes; and
d) purifying the protein from the protein-decorated liposomes.

2. The method of claim 1 wherein the means for binding of the liposomes to the protein comprises a surface moiety on the liposomes that binds to the protein.

3. The method of claim 2 wherein the means for protein binding to the liposomes comprises a moiety on the protein that binds to the liposomes.

4. The method of claim 3 wherein the moiety is added to the protein or the moiety is expressed with the protein.

5. The method of claim 1 wherein the binding is reversible, irreversible, non-covalent or covalent.

6. The method of claim 5 wherein the non-covalent means is selected from nickel-NTA-6×His tag binding, streptavidin/biotin binding, conjugate antibody binding, binding between complementary protein fragments, hybridization of complementary DNAs or RNA and binding to DNA or RNA aptamers.

7. The method of claim 6 wherein the protein comprises a His tag and the liposomes comprise a nickel-NTA moiety.

8. The method of claim 5 wherein the covalent means is selected from click chemistry and spytag/spycatcher chemistry.

9. The method of claim 1 wherein the separability is selected from a change in density of the liposomes associated with protein binding, a change in the surface charge of the liposomes, a change in the stiffness or shape of the liposomes, and a change in the fluorescence, luminescence, light absorbance or light emission of the liposomes.

10. The method of claim 1 wherein the liposomes are freeze-dried liposomes and rehydrated before use.

11. The method of claim 1 wherein the liposomes comprise phospholipids, cholesterol, conjugated phospholipids, small molecule adjuvants such as QS21 and saponins, cationic lipids internal or external to the liposome, and any combination thereof.

12. The method of claim 1 wherein the isolating is achieved by gravity centrifugation, density centrifugation, extraction, countercurrent distribution, dialysis, chromatography, precipitation, washing or any combination thereof.

13. The method of claim 1 wherein the protein is separated from the protein-decorated liposomes by proteinase cleavage, competitive binding, or a change in pH, ion content or strength, temperature or light exposure.

15. The method of claim 14 where the separated protein is purified from the liposomes by gel permeation chromatography, centrifugation, dialysis, filtration, precipitation, chromatography, or any combination thereof.

16. The method of claim 1 wherein the protein is a vaccine antigen, an enzyme, an antibody chain, a signal protein such as insulin and human growth hormone, a viral coat protein or a structural protein such as collagen.

17. A protein made by the method of claim 15.

18. A method for the preparation of protein-decorated liposomes comprising the steps of:
a) providing the protein in the presence of liposomes, wherein the protein comprises means for binding to the liposomes and the liposomes comprise means for binding to the protein, and wherein the protein binds to the liposomes forming protein-decorated liposomes, and wherein the binding of the protein to the liposomes enables the separability of the protein-decorated liposomes; and
b) isolating the protein-decorated liposomes.

19. A protein-decorated liposome made by the method of claim 18.

20. The method of claim 18 wherein the protein is a vaccine antigen.
